(19) United States
(12) Patent Application Publication
(10) Pub. No.: US 2025/0179398 A1
(43) Pub. Date: Jun. 5, 2025

(54) OVERBASED CALCIUM CARBOLXYLATES AND SULFONATES AND PROCESS FOR MAKING SAME

(71) Applicant: DELTA SPECIALTIES, 6th of October City (EG)

(72) Inventors: Eslam MOUSTAFA, 6th of October City (EG); Ali GAMAL, 6th of October City (EG)

(73) Assignee: DELTA SPECIALTIES, 6th of October City (EG)

(21) Appl. No.: 18/527,938

(22) Filed: Dec. 4, 2023

(51) Int. Cl. C11D 13/10; C11D 9/00; C11D 13/26

(52) U.S. Cl. CPC C11D 13/10; C11D 9/002; C11D 9/005; C11D 13/26

(57) ABSTRACT

A process comprising at least the steps of: (a) preparing a first mixture comprising at least one non-polar organic solvent, at least one first polar organic solvent and a catalyst, (b) preparing a second mixture comprising at least one of: C7 to C25 carboxylic acids, optionally alkyl benzene sulfonic acids and C2 to C6 carboxylic acids, (c) adding an amount of a calcium base to the first mixture to obtain a third mixture, (d) adding the second mixture of acids of step (b) to the third mixture of step (c) to obtain a fourth mixture; and carbonating the fourth mixture of step (d) by adding therein a given amount of an organic carbonate, wherein in step (c), the added amount of the calcium base allows neutralization of the carboxylic acids in step (d) and forms water as a neutralization product. The process is free of carbon dioxide injection for carbonation.

### FIELD OF THE INVENTION

The present disclosure relates generally to overbased calcium carboxylate and sulfonate soaps and process of making them, and in particular to light color, shelf-stable liquid compositions of overbased calcium carboxylate and sulfonate soaps.

### BACKGROUND OF THE INVENTION

It is well known by those skilled in the art that the overbased organic calcium salt formation mechanism involves the reaction of molar excess of calcium hydroxide or calcium oxide with organic acids to form a calcium salt. Then, the overbasing stage, includes the reaction of excess calcium hydroxide or calcium oxide with carbon dioxide to form calcium carbonate. During this reaction the organic calcium salt is adsorbed onto the surface of the colloidal calcium carbonate to form stable micelles. In order to obtain overbased calcium sulphonate or carboxylate salts, suitable organic acids include substituted or unsubstituted aliphatic, cycloaliphatic, aromatic acids, comprising sulphonic or carboxylic acids are introduced.

More specifically, the overbased calcium carboxylate soaps in solution were prepared by neutralizing calcium base in at least one aliphatic carboxylic acid and carbonating the at least one aliphatic carboxylic acid in the presence of a non-polar hydrocarbon solvent and a polar organic solvent as a promoter under controlled temperature conditions. Carbonating the overbased mixture is performed by bubbling carbon dioxide gas through the mixture.

U.S. Pat. No. 4,954,272 A, which is entirely incorporated herein by reference, describes a process for producing an overbased oil soluble calcium sulfonate by indirect approach i.e., diluting a neutral calcium sulfonate with a hydrocarbon solvent and a lower alkanol, then CaO, Ca(OH)₂ and H₂O was added in various predetermined molar ratios. Overbasing was performed by passing CO₂ gas into the sulfonate mixture at temperature from 34° C. to about 77° C. After completing the carbonation process diluent oil was added and then the product was filtered, and the hydrocarbon solvent was stripped to obtain the oily overbased calcium sulphonate product.

EP 2,599,766 A1, which is entirely incorporated herein by reference, describes the usage of fatty carboxylic acids to preparer the overbased calcium carboxylate salt by i.e., C₁₀-C₂₂ linear aliphatic carboxylic acids react with calcium hydroxide or oxide and using aliphatic hydroxy containing compound i.e., C₁-C₈ alcohols, glycols, glycol ethers as promoter. Plasticizers were used as reaction medium to obtain VOC-free overbased calcium carboxylate salt.

CN 113549481 A, which is entirely incorporated herein by reference, discloses a preparation process of overbased calcium alkyl salicylate by reacting the alkyl salicylic acids with calcium hydroxide in presence of C₁-C₄ alcohols, an acid and/or acid salt type auxiliary accelerator, and an auxiliary surfactant. The reaction medium is hydrocarbon solvent oil, to obtain overbased calcium alkyl salicylate with high yield, does not contain free phenol.

U.S. Pat. Nos. 9,828,487 B2 and 10,407,558 B2 (Moustafa et al.) by the applicant discloses respectively shelf-stable, light color liquid compositions of overbased calcium carboxylate soaps, and processes for making them, each of which is incorporated herein by reference in its entirety. These liquid compositions have light colors and excellent heat stability. They can be used as anticorrosion/antirust additives, and paint dryers and stabilizers, in paints, coatings and lubricants, as well as other applications.

Moreover, the preparation process of overbased organic calcium salts are described by several patents, including; US 3,36,5396 A, JP H08510213A, U.S. Pat. No. 5,830,832 A and EP 0949322 B1 using various procedures and compositions, each of which is incorporated herein by reference in its entirety.

However, all of the previous art carries the carbonatation by injecting carbon dioxide gas. The use of CO₂ bubbling can be a problem due to use of high pressures for injection that poses a safety hazard and complicates manufacturing process. In addition the difficulty of gas and solid reaction between carbon dioxide gas and solid calcium hydroxide/oxide represents a difficulty and necessitates the usage of big excess of CO₂ to ensure reaction completion.

Therefore, there is a need for a new process for the making of overbased calcium carboxylate soaps without the use of carbon dioxide that addresses the shortcomings of the art.

### SUMMARY OF THE INVENTION

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

According to a first aspect, the invention is related to a process for preparation of an overbased calcium carboxylate or calcium sulfonate soap, the process comprising at least the following steps: (a) preparing a first mixture comprising at least one non-polar organic solvent, at least one first polar organic solvent and a catalyst; (b) preparing a second mixture comprising at least one of: between 20 wt. % and 90 wt. % of C7 to C25 carboxylic acids, which are optionally branched on carbon 2; between 1 wt. % and 20 wt. % of C2 to C6 carboxylic acids, and optionally between 5 wt. % and 90 wt. % of alkyl benzene sulfonic acids; the wt. % being by weight of total acid; (c) adding an amount of a calcium base to the first mixture of step (a) to obtain a third mixture; (d) adding the second mixture of acids of step (b) to the third mixture of step (c) to obtain a fourth mixture; and (e) carbonating the fourth mixture of step (d) by adding therein a given amount of an organic carbonate; wherein in step (c), the added amount of the calcium base allows neutralization of the carboxylic acids in step (d) and form water as a neutralization product; and wherein the process is free of carbon dioxide injection for carbonation.

According to a preferred embodiment, the process further comprises before or after step (e): removing water, at least in part, formed as the neutralization product from said fourth mixture to obtain a dry mixture, preferably, the removing step is by distillation at a temperature from 100° C. to 120° C. and a vacuum of about 500 mmHg. More preferably, the process further comprises solvating the dry mixture with a second polar organic solvent before adding in step e) the given amount of organic carbonate.

According to a preferred embodiment, the step (d) is performed at a temperature between about 30° C. and about 60° C.

According to a preferred embodiment, the step (e) of carbonation is performed at a temperature between about 50° C. and about 60° C.

According to a preferred embodiment, the process further comprises over basing the fourth mixture before step (e) by adding therein another calcium base dispersed in a third polar organic solvent.

According to a preferred embodiment, the calcium base used in step (c) comprises calcium oxide or calcium hydroxide.

According to a preferred embodiment, the calcium base used in step (c) comprises calcium oxide.

According to a preferred embodiment, the catalyst in step (a) comprises metal oxides or zinc carboxylates.

According to a preferred embodiment, the catalyst in step (a) comprises zinc octanoate with about 16 wt. % of metal content.

According to a preferred embodiment, the C7 to C25 carboxylic acids comprises neodecanoic acid, isononanoic acid, 2-ethylhexanoic acid, neoheptanoic, neononanoic acid, or a mixture thereof.

According to a preferred embodiment, an alkyl benzene sulfonic acid is used in step (b).

According to a preferred embodiment, the alkyl benzene sulfonic acid is dodecylbenzene sulfonic acid.

According to a preferred embodiment, the C2 to C6 carboxylic acids comprise acetic acid, propionic acid, butyric acid, isobutyric acid, pentanoic acid, valeric acid, isopentanoic acid, isohexanoic acid, neohexanoic acid or mixtures thereof.

According to a preferred embodiment, the non-polar organic solvent comprises: at least one of hexane, kerosene, naphtha, benzene, toluene, ethylbenzene, or xylene, or the non-polar organic solvent comprises: a mixture of paraffinic hydrocarbons of mineral or synthetic origin.

According to a preferred embodiment, the polar organic solvent comprises C1 to C6 alcohols, glycols, glycol ethers or mixtures thereof.

According to a preferred embodiment, the polar organic solvent comprises one or more amines or a mixture of alcohols and/or amines.

According to a preferred embodiment, the organic carbonate comprises dimethyl carbonate or propylene carbonate.

According to another aspect, the invention is related to an overbased calcium carboxylate or sulfonate soap as prepared by the process as described herein.

Advantageously, the process as disclosed herewith allows carbonation without injecting carbon dioxide (CO₂) as a gas. Instead, the process employs organic carbonates in liquid phase, which result in a better, safer and more sustainable and environmentally friendly process.

Other and further aspects and advantages of the present invention will be better understood upon reading of the illustrative embodiments about to be described and various advantages not referred to herein will occur to one skilled in the art upon employment of the invention in practice.

### DETAILED DESCRIPTION OF PREFERRED EMBODIMENTS

Novel carbonic acid esters and their salts will be described hereinafter. Although the invention is described in terms of specific illustrative embodiments, it is to be understood that the embodiments described herein are by way of example only and that the scope of the invention is not intended to be limited thereby.

The terminology used herein is in accordance with definitions set out below.

As used herein % or wt. % means weight % unless otherwise indicated. When used herein % refers to weight % as compared to the total weight percent of the phase or composition that is being discussed.

By “about”, it is meant that the value of weight %, time, temperature or the like can vary within a certain range depending on the margin of error of the method or device used to evaluate such weight %, time, or temperature. A margin of error of 10% is generally accepted.

By “light color”, it is meant a solution or liquid composition having a clear yellow or amber color.

By “shelf-stable”, it is meant a solution or liquid composition being capable of storage in a closed container at room temperature for at least 90 days without suffering visible gelatization, clotting or precipitation.

The inventors have surprisingly discovered that use of organic carbonates (esters of carbonic acid), including linear carbonates (from alcohols) or cyclic carbonates (from diols) in liquid phase has significant advantages over high pressure CO₂ gas injection for producing liquid compositions of overbased calcium carboxylate and calcium sulfonate soaps.

A major advantage associated with the use of organic carbonates instead of high pressure CO₂ gas injection is in terms of the manufacturing process and specifically from the reaction thermodynamics perspective in that a liquid-solid reaction is more favorable than a gas-solid reaction (i.e., of high pressure CO₂ gas injection). That also translates into a more efficient control over the pressure and less safety hazards due to operation at lower pressure (compared to CO₂ injection). Moreover, organic carbonates in liquid phase showed a great industrial potential as a more sustainable and environmental friendly reagent compared to high pressure CO₂ gas. Moreover, the process that relies on CO₂ injection always needs excess amount of CO₂ to ensure a high level of conversion and yield. This is not the case with of organic carbonates as no excess amount of reactants are needed.

Other advantages are in terms of the resulting product which include higher degree of basicity, longer shelf stability, and improved clarity.

According to a preferred embodiment, the present disclosure relates to a process of manufacturing liquid compositions overbased calcium organic soaps, wherein the process comprises:
dispersing the basic calcium powder in an appropriate mixture of polar and non-polar organic solvents;
providing long chain (C₇-C₂₅) aliphatic Carboxylic acids (20-80% by weight to total acids), optionally (C₄-C₂₅) alkyl benzene sulfonic acids (5-90% by weight to total acids), and short chain (C₂-C₆) carboxylic acids (1% to 20% by weight to total acids) or mixtures thereof, to neutralize a part of the basic calcium powder;
heating the overbased mixture to a temperature between 40° C. and 80° C.;
carbonating the overbased mixture with an appropriate amount of organic carbonates while maintaining the temperature controlled;
heating the carbonated product to a temperature high enough to reduce the water content in the carbonated product to 0.1% by weight or less;
adding an adequate amount of polar solvent to reduce the viscosity, improve the filtration and sustain the stability; and
Catalyst may comprise metal oxides or zinc carboxylates of (C₂-C₂₀) carboxylic acids, such as zinc bis(2-ethylhexanoate).

The long chain from C7 to C25 carboxylic acids, which are preferably branched on carbon 2, may be selected from the group consisting of neodecanoic acid, isononanoic acid, 2-ethylhexanoic acid, neoheptanoic, neononanoic acid, dodecylbenzene sulfonic acid, and a mixture thereof.

The short chain C2 to C6 carboxylic acids are preferably selected from the group consisting of acetic acid, propionic acid, butyric acid, isobutyric acid, pentanoic acid, valeric acid, isopentanoic acid, isohexanoic acid, neohexanoic acid and mixtures thereof.

The non-polar organic solvent preferably contains at least one of the following: hexane, kerosene, naphtha, benzene, toluene, ethylbenzene or xylene, or a mixture of paraffinic hydrocarbons of mineral or synthetic origin, such as mineral spirits. Most preferably it containing a low proportion of not more than 30% by weight of aromatic and/or naphthenic hydrocarbons. This organic hydrocarbon liquid represents about 10% to 70% by weight of the total formulation.

The polar organic solvent used in the composition are preferably C1 to C6 alcohols, such methanol, 1-butanol, 2-butanol, or glycols, such as ethylene glycol, propylene glycol, diethylene glycol, dipropylene glycol and triethylene glycol. It can also be a glycol ether, such as ethylene glycol monobutyl ether, diethylene glycol monobutyl ether, propylene glycol monomethyl ether and dipropylene glycol monomethyl ether. It can also be a mixture of polar alcohols and/or glycols or a mixture of polar alcohols and/or glycol ethers. It can also be amines, for example, aniline, phenylenediamine, dodecylamine and mixtures thereof. It can also be a mixture of alcohols and/or amines, for example a mixture of methanol and aqueous ammonia.

Most preferably, the polar organic solvent comprises methanol, glycols, glycol ethers or mixtures thereof. If the polar organic solvent utilized is a single compound, it should preferably have a boiling point which at least 120° C. When the polar organic solvent is comprised of two or more different polar organic compounds, one or more of those polar compounds will preferably have a boiling point which is below 120° C. In the case of a polar organic solvent which is comprised of multiple different polar organic compounds, it is preferred that at least one of the compounds have a boiling point which is 120° C. or higher.

An appropriate amount of organic carbonates is used for carbonating the overbased mixture. Non-liming examples of organic carbonates comprises: Acyclic organic carbonates such as dimethyl carbonate (CH₃OCOOCH₃), Diethyl carbonate (C₂H₅OCOOC₂H₅), Di-isopropyl carbonate (CH₃)₂CHOCOOCH(CH₃)₂, and Dibutyl carbonate (C₄H₉OCOOC₄H₉), and cyclic organic carbonates such as Ethylene carbonate (C₃H₄O₃), propylene carbonate (C₄H₆O₃), and Vinylene carbonate (C₃H₂O₃).

### EXAMPLES

### Example 1

An over-based calcium soap is prepared in 1 Liter four inlets round glass reactor using an efficient mechanical stirrer, an electric heating system and a Dean stark distillation apparatus. 112.5 g of mineral Spirit, 18 g of dipropylene glycol monomethyl ether, 0.2 g zinc octanoate (16% metal content), 40 g of methanol and 10 g of neodecanoic acid are introduced into the reactor, and the stirring speed is adjusted at 350 RPM. 50 gm calcium oxide 97% are slowly added to assure the good dispersion of powder in the liquids mixture. Using a 500 ml dropping funnel, an acid mixture of 48.07 g of neodecanoic acid and 14 g of propionic acid is added drop-wise within one hour keeping the temperature within 30-60° C., 60 g of dimethyl carbonate is then added drop-wisely within 30 min while keeping the temperature within 50-60° C. The reaction mixture is then heated to 120° C. under a vacuum of 500 mm Hg for 30 min to make sure that all the methanol and water are removed. After cooling to 80° C., 20 g of methoxypropanol are added. The obtained product is filtered using a filtration vacuum pump to obtain yellowish clear liquid product, which was then diluted to adjust the calcium content to 10% (w/w).

### Example 2

An over-based calcium soap is prepared in a 1 Liter four-inlet round glass reactor using an efficient mechanical stirrer, an electric heating system and a Dean stark distillation apparatus. 148.53, g of toluene, 24.8 g of dipropylene glycol monomethyl ether, 0.2 g of zinc octanoate (16% metal content) are added into the reactor. The stirring speed is adjusted at 350 rpm. 50 g of calcium oxide 97% are added slowly with good mixing to assure the dispersion of the powder in the liquids mixture. Using a 500 ml dropping funnel, an acid mixture of 35.70 g of isononanoic acid, 25 g of 2-ethylhexanoic acid and 7.7 g of propionic acid were added drop-wise within one hour keeping the temperature within 30-60° C. The mixture was then heated to 110° C. followed by a 500 mm Hg vacuum for 30 min to assure removing the neutralization water. The reactor is then cooled to 80° C. followed by the addition of 30 g of mineral Spirit, 49 g of petroleum ether, 40 g of methanol, 5.3 g of demineralized Water. After good mixing and dispersing the added powder, 60 gm dimethyl carbonate is added drop-wise over 30 min while maintaining the reaction temperature at around 50° C. The composition is then heated to 120° C. within 1 hour followed by a vacuum of around 500 mmHg for 30 min to remove the methanol and water. After cooling to 80° C., 20 g methoxypropanol are added. The product was diluted to a calcium content of 10% (w/w) and filtered using a filtration vacuum pump to obtain a clear yellowish easily filtered liquid product.

### Example 3

The same procedure of Example 2 is repeated except that the acid mixture is composed of 45.83 g of neodecanoic acid, 3.85 g of propionic acid and 62.28 dodecylbenzene sulfonic acid. A clear slightly reddish filterable product is obtained.

### Example 4

The same procedure and composition of Example 3 are repeated except that methanol is not added. The obtained product was clear, yellowish, filtrable, and slightly viscous compared to the product of Example 3.

### Example 5

The same procedure of Example 2 is repeated except that the acid mixture comprises: 50 g of 2-ethylhexanoic acid, 10 g of neodecanoic acid, 5.8 g of propionic acid, and 8.5 g of dodecylbenzenesulfonic acid. A clear yellowish filterable product is obtained.

### Example 6

The same procedure of Example 1 is repeated except that methanol is not used and the acid mixture is composed of: 37.43 g of neodecanoic acid, 25 g of 2-ethylhexanoic acid, and 7.7 g of propionic acid. Further, the dimethyl carbonate was premixed with 5.3 g of demineralized water before the addition. The obtained product was a clear light yellow liquid.

### Example 7

The same procedure of example 2 was repeated except that the used acid mixture is 41.25 g of isononanoic acid, 30 g of 2-ethylhexanoic acid and 9.24 g of propionic acid and the dimethyl carbonate was replaced by 35 g of propylene carbonate. A clear yellowish product was obtained.

### Example 8

The same procedure of example 6 was repeated except introducing 40 g of methanol before adding the acids and replacing the dimethyl carbonate with the propylene carbonate.

### Example 9

The same procedure of example 6 was repeated except that the acid ratio was as the following; 35.66 g of 2-ethylhexanoic acid, 10 g of neodecanoic acid, 3.8 g of propionic acid 51.90 g of dodecylbenzenesulfonic acid. A clear, slightly reddish, and easily filtered product was obtained.

### Comparative Example 10

The same procedure as in Example 1 except that the dimethyl carbonate is removed and carbon dioxide gas is injected directly after the acid mixing is completed. The gas injection was injected at a rate of 40 liters/hour for one and half hour. The obtained product was highly viscous and unable to be filtered.

### Comparative Example 11

The dimethyl carbonate stage was replaced with injection of carbon dioxide at a rate of 40 litres per hour for one and a half hour, using the same process and ingredients as in Example 1 with the exception of the same acid mixture as in Example 6. The obtained product was not filterable, had bad yield and very cloudy.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

What is claimed is:

1. A process for preparation of an overbased calcium carboxylate or calcium sulfonate soap, the process comprising at least the following steps:
(a) preparing a first mixture comprising at least one non-polar organic solvent, at least one first polar organic solvent and a catalyst;
(b) preparing a second mixture comprising at least one of:
between 20 wt. % and 90 wt. % of C7 to C25 carboxylic acids, which are optionally branched on carbon 2;
between 1 wt. % and 20 wt. % of C2 to C6 carboxylic acids,
and
optionally between 5 wt. % and 90 wt. % of alkyl benzene sulfonic acids;
the wt. % being by weight of total acid;
(c) adding an amount of a calcium base to the first mixture of step (a) to obtain a third mixture;
(d) adding the second mixture of acids of step (b) to the third mixture of step (c) to obtain a fourth mixture; and
(e) carbonating the fourth mixture of step (d) by adding therein a given amount of an organic carbonate;
wherein in step (c), the added amount of the calcium base allows neutralization of the carboxylic acids in step (d) and forms water as a neutralization product; and
wherein the process is free of carbon dioxide injection for carbonation.

2. The process of claim 1, further comprising before or after step (e):
removing water, at least in part, formed as the neutralization product from said fourth mixture to obtain a dry mixture.

3. The process of claim 2, wherein the removing step is by distillation at a temperature from 100° C. to 120° C. and a vacuum of about 500 mmHg.

4. The process of claim 2, further comprising:
solvating the dry mixture with a second polar organic solvent before adding in step e) the given amount of organic carbonate.

5. The process of claim 1, wherein the step (d) is performed at a temperature between about 30° C. and about 60° C.

6. The process of claim 1, wherein the step (e) of carbonation is performed at a temperature between about 50° C. and about 60° C.

7. The process of claim 1, further comprising before step (e):
over basing the fourth mixture by adding therein another calcium base dispersed in a third polar organic solvent.

8. The process of claim 1, wherein the calcium base used in step (c) comprises calcium oxide or calcium hydroxide.

9. The process of claim 8, wherein the calcium base used in step (c) comprises calcium oxide.

10. The process of claim 1, wherein the catalyst in step (a) comprises metal oxides or zinc carboxylates.

11. The process of claim 1, wherein the catalyst in step (a) comprises zinc octanoate with about 16 wt. % of metal content.

12. The process of claim 1, wherein the C₇ to C₂₅ carboxylic acids comprises neodecanoic acid, isononanoic acid, 2-ethylhexanoic acid, neoheptanoic, neononanoic acid, or a mixture thereof.

13. The process of claim 1, wherein an alkyl benzene sulfonic acid is used in step b).

14. The process of claim 13, wherein the alkyl benzene sulfonic acid is dodecylbenzene sulfonic acid.

15. The process of claim 1, wherein the C₂ to C₆ carboxylic acids comprise acetic acid, propionic acid, butyric acid, isobutyric acid, pentanoic acid, valeric acid, isopentanoic acid, isohexanoic acid, neohexanoic acid or mixtures thereof.

16. The process of claim 1, wherein the non-polar organic solvent comprises:
at least one of hexane, kerosene, naphtha, benzene, toluene, ethylbenzene, or xylene, or
a mixture of paraffinic hydrocarbons of mineral or synthetic origin.

17. The process of claim 1, wherein the polar organic solvent comprises C₁ to C₆ alcohols, glycols, glycol ethers or mixtures thereof.

18. The process of claim 1, wherein the polar organic solvent comprises one or more amines or a mixture of alcohols and/or amines.

19. The process of claim 1, wherein the organic carbonate comprises:
acyclic organic carbonates selected from the group consisting of: dimethyl carbonate (CH₃OCOOCH₃), diethyl carbonate (C₂H₅OCOOC₂H₅), di-isopropyl carbonate (CH₃)₂CHOCOOCH(CH₃)₂, and dibutyl carbonate (C₄H₉OCOOC₄H₉), or
cyclic organic carbonates selected from the group consisting of ethylene carbonate (C₃H₄O₃), propylene carbonate (C₄H₆O₃), and vinylene carbonate (C₃H₂O₃).

20. An overbased calcium carboxylate or sulfonate soap as prepared according to the process of claim 1.
